(19) United States
(12) Patent Application Publication
(10) Pub. No.: US 2003/0103960 A1
(43) Pub. Date: Jun. 5, 2003

(54) Sealant and bone generating product

(76) Inventors: Pierre Philippart, Brussels (BE); Michele Brasseur, Brussels (BE)

Correspondence Address: LEYDIG VOIT & MAYER, LTD, 700 THIRTEENTH ST. NW, SUITE 300, WASHINGTON, DC, 20005-3960, US

(21) Appl. No.: 10/176,401

(22) Filed: Jun. 21, 2002

Related U.S. Application Data
(63) Continuation-in-part of application No. 09/469,302, filed on Dec. 22, 1999, now abandoned.

(30) Foreign Application Priority Data
Dec. 20, 2000 (WO) PCT/BE00/00152
Aug. 16, 2000 (WO) PCT/BE00/00094

(51) Int. Cl. A61K 38/48; C12P 21/06

(52) U.S. Cl. 424/94.64; 435/68.1

(57) ABSTRACT

The sealant and bone generating product comprises a coagulated matrix of platelet rich plasma with a recombinant compound for generating thrombin mixed with two different phospholipids. The bone generating product comprises an effective amount of calcium containing compound dispersed in the matrix for inducing the formation of bone.

The present application is a Continuation in part of co-pending International application No. PCT/BE00/00152, with an international filing date of Dec. 20, 2000, published in English under PCT Article 21(2) on Jun. 28, 2001 which claims the benefit of the priority of co-pending application U.S. Ser. No. 09/469,302 filed on Dec. 22, 1999, as well as of International application PCT/BE00/00094 filed on Aug. 16, 2000.

### FIELD OF THE INVENTION

The invention relates to a bone generating product comprising a coagulated matrix of platelet rich plasma with a recombinant compound for generating thrombin in presence of at least a phospholipid, and an effective amount of calcium containing compound dispersed in the matrix for inducing the formation of bone.

### THE PRIOR ART

Many researches have been made for the preparation of bone substitute or implant.

For the preparation of bone substitute or implant, it is known to treat human bone by chemicals for destroying prions. The so treated human bone acts as a porous matrix suitable for the growth of cells after its implant.

It has also been proposed to prepare artificial matrix or sponge from collagen containing material and to use said matrix or sponge as bone substitute.

Example 4 of U.S. Pat. No. 5,733,545 discloses the preparation of clot from a mixture containing a plasma-buffy coat concentrate and ground dry bone or from a plasma-buffy coat concentrate and CaCl₂, said latter compound being used for ensuring the coagulation of the mixture. In said example 4, it is stated that the chelation of the plasma-buffy coat concentrate containing ground dry bone is possibly due to the presence of calcium from the solid bone. In said example, it is clearly stipulated that the use of thrombin is a cause of patient complications.

However, the bone substitute obtained by mixing a plasma-buffy coat concentrate and ground dry bone was not suitable for the bone generation.

For inducing bone repair, Friadent is commercializing the product “PEPGEN 15” and “pepgen 15 Flow” which consist of a synthetic resorbable matrix containing a synthetic amino acid peptide of formula GTPGPQGIAGQRGVV, and inorganic particles containing calcium phosphate and having a mean particle size lower than 750 μm.

This product enables good histologic regeneration in 2/4 cases evaluated.

It has now been found that it was possible to ensure an increased histologic generation, by using the synthetic amino acid peptide of formula GTPGPQGIAGQRGVV, and inorganic particles containing calcium phosphate and having a mean particle size lower than 750 μm, in the technology disclosed in the pending application U.S. Ser. No. 09/469302.

In the present invention, the recombinant tissue factor with or in presence of a phospholipid acts as a recombinant thrombin generating product, but also as a means to induce platelet degranulation and as a means for liberating growth factor present in the platelet. Preferably, at least a part of the calcium phosphate containing compound is made from bone particles. For example, at least 30%, preferably at least 50% by weight of the calcium containing compound is made from bone particles, preferably of not denatured bone particles. The presence of the bone particles in the bone generating product of the invention is considered as improving the generation of bone, as said bone particles contain bone morphogenic proteins as well growth factors for directing the tissue factor to produce bone. It is assumed that the excellent bone generation obtained by implanting the bone generating product of the invention to patient is due to the presence of the various factors (growth factors, etc.) present in the platelet rich plasma and of calcium containing compound(s) (preferably bone particles). The presence of recombinant tissue factor is also preferred. It is assumed that the recombinant tissue factor induces a protein containing matrix, factor suitable for inducing and accelerating the formation of bone in presence of calcium containing compound. When using substantially only platelet rich plasma, bone particles and a recombinant thrombin generating compound, it is assumed that the different factors present in the product of the invention act substantially as in the human body (the ratio of one factor with respect to another being substantially equal to the said ratio in the human body), whereby improving the generation of bone.

The presence of peptide of formula GTPGPQGIAGQRGVV and inorganic particles containing calcium phosphate and having a mean particle size lower than 750 μm in the PRP matrix of the invention seems to further improve and/or increase the bone formation by increasing the cell binding on the solid inorganic calcium phosphate particles, and therefore the cell migration and proliferation, as well as by increasing the cell differentiation.

Tests made by Applicants on four volunteers have shown (histological analysis) a bone generation in less than 1 month for all the patients, without any adverse reactions, such as inflammation, local reaction, tissue reaction, separation of particles, etc.

### BRIEF DESCRIPTION OF THE INVENTION

The invention first relates to a method for the preparation of a sealant, in which a fibrinogen containing solution (such as PPP, but preferably PRP) is contacted with a recombinant compound for generating thrombin (such as a tissue factor, preferably having no membrane binding sequence) in presence of at least a phospholipid (preferably at least two different phospholipids), at least a buffer and at least an antibiotic. The sealant is advantageously formed on the bleeding organ to be sealed.

The invention relates also to a kit for the preparation of a sealant by contacting a fibrinogen containing solution with a recombinant compound for generating thrombin in presence of at least a phospholipid, at least a buffer and at least an antibiotic, said kit comprising at least:

one system selected from the group consisting of a vial containing a recombinant compound for generating thrombin, at least two different phospholipids, a buffer and an antibiotic; two distinct vials, a first containing a recombinant compound for generating thrombin and at least two different phospholipids, while the second contains a buffer and an antibiotic; three distinct vials, a first containing a recombinant compound for generating thrombin and at least two different phospholipids, the second containing a buffer and the third containing an antibiotic; and a system consisting of a vial system containing a recombinant compound for generating thrombin, at least two different phospholipids and a buffer, and an antibiotic formulation for oral administration.

The kit comprises advantageously a container for receiving the fibrinogen containing solution to be mixed with the recombinant product and the phospholipid(s). The kit may also comprises means for ensuring the mixing the fibrinogen containing solution with the recombinant product and the phospholipids, as well as possibly means for applying the sealant or the mixed solution suitable to form the sealant, on a bleeding part of an organism. The application means can be a means suitable for spraying the mixed solution, before the complete gelling of the sealant.

The invention relates further to a bone generating product comprising a coagulated matrix of platelet plasma with a recombinant compound for generating thrombin in presence of at least one phospholipid, and an effective amount of calcium containing compound dispersed in the said matrix for inducing the formation of bone. The calcium containing compound is advantageously inorganic particles containing calcium phosphate, such as bone particles, hydroxyapatite particles, synthetic hydroxyapatite particles, said particles being advantageously bound or coated with an amino acid peptide, preferably a synthetic amino acid peptide.

The invention relates therefore also to a method for preparing such a bone generating product.

Further object of the invention are:

the use of a recombinant compound for generating thrombin in mixture with at least one phospholipid for the preparation of a bone generating product from a mixture of a fibrinogen containing solution and an effective amount of calcium containing compound for inducing bone generation;

a mixture containing a recombinant compound for generating thrombin, at least one phospholipid, and a calcium containing compound, the weight ratio calcium from the calcium containing compound/recombinant compound for generating thrombin being greater than 0.5;

a mixture containing a recombinant compound for generating thrombin, at least one phospholipid, and at least one antibiotic, the weight ratio antibiotic/recombinant compound for generating thrombin being greater than 1;

A kit for the preparation of a bone generating composition prepared by contacting a fibrinogen containing solution with a recombinant compound for generating thrombin in presence of at least a phospholipid, and inorganic particles containing calcium phosphate and having a mean particle size lower than 750 μm, said kit comprising at least one system selected from the group consisting of a vial containing a recombinant compound for generating thrombin, and inorganic particles containing calcium phosphate and having a mean particle size lower than 750 μm, two distinct vials, a first containing a recombinant compound for generating thrombin, while the second contains the inorganic particles containing calcium phosphate and having a mean particle size lower than 750 μm;

a method for generating bone to a patient in need, said method comprising the step of applying at the place where bone has to be generated a bone generating product comprising a coagulated matrix of fibrinogen containing solution with a recombinant compound for generating thrombin in presence of at least a phospholipid, and inorganic particles containing calcium phosphate and having a mean particle size lower than 750 μm.

In the invention, the thromboplastin or tissue factor is preferably adapted for not being associated in natural way with the phospholipids, but is mixed with the phospholipids.

### DESCRIPTION OF EMBODIMENTS OF THE INVENTION

The bone generating product of the invention comprises a coagulated matrix of fibrinogen containing solution, preferably a platelet rich plasma, with a recombinant compound for generating thrombin, said matrix comprising at least a phospholipid and an effective amount of calcium containing compound for generating bone dispersed in the matrix. Preferably, the calcium containing compound is bone particles, possibly mixed with other calcium containing compound, synthetic calcium phosphate particles, etc. Preferably, at least 30% by weight, advantageously at least 50% by weight of the calcium containing compound consists of bone particles. Examples of calcium containing compounds are: CaCl₂, β-tricalcium phosphate, bone particles (denatured bone), bone particles (not denatured bone), apatite, aspidine, calcium sulfate, calcium carbonate, hydroxyapatite, hydroxyapatite (from coral reef), calcium gluconolactate, calcium gluconate, calcium lactate, calcium glutonate and mixtures thereof.

Advantageously, the recombinant compound for generating thrombin is a recombinant thromboplastin or a recombinant tissue factor, such as a recombinant tissue factor having no membrane binding sequence or having only sequence of the extracellular domain.

Preferably, the bone generating product of the invention comprises two or more than two different phospholipids.

According to an advantageous embodiment, the recombinant compound for generating thrombin is combined, preferably mixed, with one, preferably at least two different phospholipids.

According to an preferred embodiment, the recombinant compound for generating thrombin is combined, preferably mixed, with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof Preferably, the recombinant compound for generating thrombin is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof, the fatty acid side chain being selected from the group consisting of fatty acid chains with at least one double bond and with 6 to 24 carbon atoms, most preferably with 16 to 18 carbon atoms.

According to a most preferred embodiment, the recombinant compound for generating thrombin is combined, preferably mixed, with a mixture of at least two phospholipids, a first phospholipid being selected from the group consisting of phosphatidylserine, derivatives thereof, phosphatidylserine having at least one fatty acid side chain, and mixtures therof, the fatty acid side chain of the phosphatidylserine having at least one fatty acid side chain being selected from the group consisting of fatty acid chains with at least one double bond and with 6 to 24 carbon atoms, preferably 16 to 18, while the second phospholipid is selected from the group consisting of phosphatidylcholine, derivatives thereof, phosphatidylcholine having at least one fatty acid side chain, and mixtures thereof, the fatty acid side chain of the phosphatidycholine having at least one fatty acid side chain being selected from the group consisting of fatty acid chains with at least one double bond and with 6 to 24 carbon atoms.

In the bone generating product of the invention, the bone particles are preferably bone particles selected from the group consisting of craniofacial bone particles, iliac bone particles and mixture thereof. Preferably, the bone particles are particles of not denatured bones. Advantageously, the bone particles have an average particle size comprised between 0.5 mm and 5 mm, preferably comprised between 0.5 and 3 mm, most preferably about 1 mm (average in weight). The bone particles have for example the form of chips or flakes having an average particle size comprised between 0.5 mm and 5 mm, preferably comprised between 0.5 and 3 mm, most preferably about 1 mm (average in weight). According to a possible embodiment, the bone particles consist of a mixture of denatured bone particles (for example bone particles prepared by grinding a bone that has been treated by chemical(s), by irradiation, etc. for rendering it prion free.) and of not denatured bone particles. When using some denatured bone particles, the said particles of denatured bone can have a particle size lower than 0.5 mm, as said denatured bone particles are used to add adding some calcium to the product.

The bone generating product of the invention comprises for example from 5% to 50% by volume of bone particles, advantageously from 10 to 40%, preferably from 20 to 30% by volume of bone particles. The bone particles forms preferably more than 90% by weight of the calcium containing compound present in the bone generating product of the invention.

According to an advantageous embodiment of the bone generating product of the invention, the coagulated matrix is a coagulated matrix of platelet rich plasma having a platelet concentration of 1,500,000 to 2,000,000 platelets per microlitre of the matrix forming agents, and preferably 0.05 to 5 μg thromboplastin in dry form per microlitre of the matrix forming agents. The platelet concentration is a concentration adapted for ensuring the viability of the platelets at 37° C.

In order to avoid as much as possible complication and in order to improve as much as possible the graft of the bone generated on the natural bone of a patient, the platelet rich plasma used is prepared from the plasma of the patient and/or from a histocompatible plasma (i.e. immunological histocompatibility), while the bone particles are prepared from a bone of the patient and/or from histocompatible bone(s) (i.e. immuno histocompatibility).

The bone generating product of the invention can possibly contain further components or additives, such as growth factor (PDGFAA,PDGFAB,PDGFBB, superfamily BTGF and family of BMP, such as BMP-1, etc.), gene coding BMP and/or BTGF, steric factors, calcium containing compounds, drugs, fatty acids, antibiotics or mixtures of antibiotics (preferably compound(s) having an anti osteoclasts effect, such as antibiotics of the tetracyclin group, Vibramycin®, Doxycycline®, Minocycline, Minocin® (Wyeth-Lederlee), and mixtures of compound(s) having an anti osteoclasts effect with another antibiotic(s), such as macrolide, penicillin based compounds, etc.), bactericide, virucide, fibrinogen, compounds inducing the formation of a matrix, buffer, zwitterionic buffer system at physiological pH, etc. and mixtures of said compounds or additives.

According to a detail of a preferred embodiment, the bone generating product contains from 0.001 to 10% by weight antibiotic or antibiotics (calculated in its dry form), advantageously from 0.01% to 5% by weight, preferably from 0.02 to 1%, for example from 0.05 to 0.4% by weight. The antibiotic is advantageously selected from the group consisting of antibiotics having an anti osteoclasts effect (more specifically antibiotics of the tetracyclin group, Vibramycin®, Doxycycline®, Minocycline, Minocin® (Wyeth-Lederlee)), mixtures of antibiotics having an anti osteoclasts effect, and mixtures of one or more antibiotics having an anti osteoclasts effect with one or more other antibiotic(s) (preferably macrolide, penicillin based compounds, etc. and mixtures thereof).

Before its gelling, the bone generating product has advantageously a pH substantially equal to the physiological pH, for example a pH comprised between 6.5 and 8, preferably about 7-7.5, pH measured at 37° C.

According to an embodiment, the bone generating product of the invention is a bone generating product comprising a coagulated matrix of fibrinogen containing solution, advantageously platelet containing solution, preferably of platelet rich plasma, with a recombinant compound for generating thrombin in presence of at least a phospholipid, a synthetic amino acid peptide of formula GTPGPQGIAGQRGVV, and inorganic particles containing calcium phosphate and having a mean particle size lower than 750 μm.

Preferred details of said embodiment are given here below.

Advantageously, in said embodiment, the calcium phosphate containing compound is selected from the group consisting of bone particles, mixture of bone particles with calcium containing additives, synthetic hydroxyapatite, and mixtures thereof. The bone particles are advantageously particles derived from non-denatured bones.

Advantageously, the recombinant compound for generating thrombin is a recombinant thromboplastin or a recombinant tissue factor.

Advantageously, the recombinant compound for generating thrombin is a recombinant tissue factor having no membrane binding sequence or having only sequence of the extracellular domain.

According to an advantageous embodiment, the recombinant compound for generating thrombin is combined, preferably mixed, with one, preferably at least two different phospholipids.

Preferably, the bone generating product further comprises at least a buffer agent and an antibiotic.

According to a detail of a preferred embodiment, the calcium phosphate containing particles have a mean size comprised between 150 μm and 500 μm.

Advantageously, the weight ratio peptide of formula GTPGPQGIAGQRGVV/calcium phosphate containing particles is comprised between 0.0001 and 0.001, advantageously between 0.00015 and 0.0005, preferably about 0.00025.

When bone is used as source for calcium phosphate containing material, the bone particles are preferably selected from the group consisting of craniofacial bone particles, iliac bone particles and mixtures thereof.

According to a specific embodiment, in which the calcium containing compound is selected from the group consisting of bone particles, mixture of bone particles with calcium containing additives, the bone particles are advantageously particles derived from non-denatured bones.

Most preferably, the calcium phosphate containing particles have substantially no sharp and pointed edge.

When using PRP for the matrix, the platelet rich plasma has advantageously a platelet concentration of 1,500,000 to 2,000,000 platelets per microlitre of the matrix forming agents.

For example, the coagulated matrix is a coagulated matrix prepared by gelling a platelet rich plasma having a platelet concentration of 1,500,000 to 2,000,000 platelets per microlitre in presence of from 1 to 200 μg (advantageously between 2 and 100 μg, preferably between 5 and 75 μg) recombinant tissue factor in dry form per ml PRP, from 1 to 4,000 μg (advantageously from 5 to 3000,μg, preferably from 50 to 2,000 μg) of phospholipids, from 0.01 to 10 g (advantageously from 0.5 to 5 g, preferably less than about 3 g) of hydroxyapatite particles per ml PRP and from 1 to 5,000 ng (advantageously from 5 to 1,500 ng, preferably from 10 to 1000 ng) peptide of formula GTPGPQGIAGQRGVV per ml PRP.

The platelet rich plasma can be prepared from the plasma of the patient (for example submitted to a prior treatment with an efficient amount of antibiotic(s)), while the bone particles are prepared from a bone of the patient.

According to a possible embodiment, the coagulated matrix is associated with a bio compatible film.

The bone generating product may further contain one or more additives selected among the group consisting of growth factors, genes encoding growth factors, calcium containing compounds, drugs, fatty acids, bactericides, virucides, compounds inducing the formation of matrixes, and mixtures thereof

Most preferably, the bone generating product contains at least an antibiotic having an anti osteoclasts effect.

The bone generating product of the invention comprises advantageously a coagulated matrix of platelet rich plasma with a recombinant compound for generating thrombin, said matrix comprising a sufficient amount of peptide of formula GTPGPQGIAGQRGVV, at least a phospholipid and an effective amount of calcium phosphate containing compound for generating bone dispersed in the matrix. Preferably, the calcium containing compound is bone particles, possibly mixed with other calcium containing compound. Preferably, at least 30% by weight, advantageously at least 50% by weight of the calcium containing compound consists of bone particles. Examples of calcium phosphate containing compounds are: β-tricalcium phosphate, bone particles (denatured bone), bone particles (non-denatured bone), hydroxyapatite, hydroxyapatite (from coral reef), and mixtures thereof, said compounds being possibly mixed with one or more calcium containing compound, such as CaCl₂, apatite, aspidine, calcium sulfate, calcium carbonate, calcium gluconolactate, calcium gluconate, calcium lactate, calcium glutonate, and mixtures thereof.

According to a possible embodiment, the bone particles consist of a mixture of denatured bone particles (for example bone particles prepared by grinding a bone that has been treated by chemical(s), by irradiation, etc. for rendering it prion free.) and of non-denatured bone particles. When using some denatured bone particles, the said particles of denatured bone can have a particle size lower than 0.5 mm, as said denatured bone particles are used to add some calcium to the product.

The bone generating product of the invention comprises for example from 5% to 50% by volume of calcium phosphate (such as bone) particles, advantageously from 10 to 40%, preferably from 20 to 30% by volume.

The invention relates also to a method for the preparation of a bone generating product comprising a coagulated matrix of fibrinogen containing solution (advantageously platelet containing solution, preferably platelet rich plasma) with a recombinant compound for generating thrombin, and solid calcium containing compounds (such as bone particles, synthetic hydroxyapatite) dispersed in said matrix, in which

a substantially homogeneous mixture is formed by mixing of fibrinogen containing solution (preferably platelet rich plasma) with an effective amount of calcium containing compound(s) for inducing the bone generation when adding to the mixture a recombinant thrombin generating compound and a phospholipid,

a recombinant thrombin generating compound and at least one phospholipid are added and mixed to the mixture of calcium containing compound (such as bone particles or hydroxyapatite) and fibrinogen containing solution (preferably platelet rich plasma), and

the mixture recombinant thrombin generating compound, platelet rich plasma, phospholipid and bone particles is kept under conditions for ensuring a coagulation of the platelet rich plasma and the formation of a bone generating matrix.

Preferably, the coagulation of the fibrinogen is carried out in presence of oxygen and substantially without stirring. The said coagulation is most preferably carried out at a temperature comprised between 35° C. and 40° C., more specifically at a temperature of about 37° C.

In the process of the invention, the recombinant compound for generating thrombin used for the coagulation is advantageously a recombinant thromboplastin or a recombinant tissue factor.

Advantageously, the recombinant compound for generating thrombin is a recombinant tissue factor, such as a recombinant tissue factor having no membrane binding sequence or having only sequence of the extracellular domain.

According to an advantageous embodiment, the recombinant compound for generating thrombin is combined, preferably mixed, with one, preferably at least two different phospholipids.

Advantageously, at least two different phospholipids are added to the mixture selected from the group consisting of mixture of recombinant thrombin generating compound, platelet rich plasma and bone particles, and mixture of platelet rich plasma and bone particles, said addition being preferably carried out when adding the recombinant thrombin generating compound.

In the process of the invention, the recombinant thrombin generating compound is advantageously combined with phospholipid, preferably with phospholipids, the said compound combined with phospholipid(s) having advantageously the form of a lyophilized product, such as a lyophilized cake, powder or granules.

According to a preferred method of the invention, the recombinant compound for generating thrombin is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof, the fatty acid side chain being advantageously selected from the group consisting of fatty acid chains with at least one double bond and with 6 to 24 carbon atoms, preferably with 16-18 carbon atoms.

According to a most preferred embodiment of the process, the recombinant compound for generating thrombin is combined with a mixture of at least two phospholipids, a first phospholipid being selected from the group consisting of phosphatidylserine, derivatives thereof, phosphatidylserine having at least one fatty acid side chain, and mixtures therof, the fatty acid side chain of the phosphatidylserine having at least one fatty acid side chain being selected from the group consisting of fatty acid chains with at least one double bond and with 6 to 24 carbon atoms, while the second phospholipid is selected from the group consisting of phosphatidylcholine, derivatives thereof, phosphatidylcholine having at least one fatty acid side chain, and mixtures thereof, the fatty acid side chain of the phosphatidycholine having at least one fatty acid side chain being selected from the group consisting of fatty acid chains with at least one double bond and with 6 to 24 carbon atoms.

Preferably, at least a part of the calcium containing compound(s) is formed by bone particles, advantageously bone particles selected from the group consisting of craniofacial bone particles, iliac bone particles and mixture thereof. The bone particles are advantageously particles of not denatured bones. Said bone particles can possibly consist of a mixture of not denatured bone particles and denatured bone particles. The bone particles have advantageously an average (by weight) particle size comprised between 0.5 mm and 5 mm, preferably comprised between 0.5 and 3 mm, most preferably of about 1 mm.

In the method of the invention, the amount of bone particles added to the platelet rich plasma corresponds for example to about 5% to 50% by volume, advantageously from 10 to 40%, preferably from 20% to 30% by volume of the mixture platelet rich plasma and bone particles.

Advantageously, the platelet rich plasma used in the method of the invention has a platelet concentration of 1,500,000 to 2,000,000 platelets per microlitre of the matrix forming agents.

Preferably, the mixture platelet rich plasma, bone particles and recombinant thrombin generating compound has a platelet concentration of 1,500,000 to 2,000,000 platelets per microlitre of the mixture without bone particles and contains 0.05 to 5 μg thromboplastin in dry form per microlitre of the mixture without bone particles.

According to a preferred method of the invention suitable for the preparation of a bone generating product for a patient, the platelet containing solution, preferably the platelet rich plasma is prepared from the plasma of the patient and/or from a histocompatible plasma and in which the bone particles are prepared from a bone of the patient and/or from a histocompatible bone.

According to a detail of a preferred method of the invention, the coagulation of the platelet rich plasma is carried out in presence of at least one antibiotic and/or at least one antibiotic is added to the mixture after the coagulation of the platelet rich plasma. The antibiotic or mixture of antibiotics can possibly be added to the bone particles and/or to the bone before its grinding and/or to the recombinant compound that generates thrombin and/or to a phospholipid. Preferably, an antibiotic or a mixture of antibiotics is mixed with the recombinant compound for generating thrombin (recombinant compound that generates thrombin), preferably with a recombinant thromboplastin.

According to a detail of a preferred embodiment, the amount of antibiotic or antibiotics added to the bone generating product or used during the coagulation of the platelet rich plasma bone generating product contains from 0.001 to 10% by weight antibiotic or antibiotics (calculated in its dry forms), advantageously from 0.01% to 5% by weight, preferably from 0.02 to 1%, for example from 0.05% to 0.4% by weight, more specifically from 0.2 to 0.3%. The antibiotic is advantageously selected from the group consisting of antibiotics having an anti osteoclasts effect (more specifically antibiotics of the tetracyclin group, Vibramycin®, Doxycycline®, minocycline, minocin® (Wyeth-Lederlee)), mixtures of antibiotics having an anti osteoclasts effect, and mixtures of one or more antibiotics having an anti osteoclasts effect with one or more other antibiotic(s) (preferably macrolide, penicillin based compounds, etc. and mixtures thereof).

Most preferably, at least one antibiotic is added to the mixture containing at least platelet rich plasma and calcium containing compound, before adding the recombinant compound that generates thrombin, but advantageously when adding the recombinant compound that generates thrombin.

According to a preferred method, the method for the preparation of a bone generating composition is a method, in which a fibrinogen containing solution (such as PPP, preferably PRP) is contacted, preferably mixed, with a recombinant compound for generating thrombin in presence of at least a phospholipid, a synthetic amino acid peptide of formula GTPGPQGIAGQRGVV, and inorganic particles containing calcium phosphate and having a mean particle size lower than 750 μm.

The following table gives the correlation between the amino acid, its one-letter code and its three letter code.

| | One-letter | Three-letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

In a preferred method of the invention, a gel (most preferably a hydrogel) is advantageously formed by the contact (preferably the mixing) of the fibrinogen containing solution with a recombinant compound for generating thrombin in presence of at least a phospholipid, a synthetic amino acid peptide of formula GTPGPQGIAGQRGVV, and said inorganic particles, whereby during the gel formation, the pH of the fibrinogen solution is kept between 6 and 8.

Preferably, the gel or coagulation of the fibrinogen is carried out in presence of oxygen and substantially without stirring. The said coagulation is most preferably carried out at a temperature comprised between 35° C. and 40° C., more specifically at a temperature of about 37° C.

According to a preferred embodiment, the gel is formed in presence of an antibiotic and at least a buffer agent.

Possible buffers are for example TRIS buffer, solution of Ringé, sodium bicarbonate, and mixture thereof.

Most preferably, the recombinant compound for generating thrombin is a recombinant thromboplastin or a recombinant tissue factor.

Advantageously, the recombinant compound for generating thrombin comprises a recombinant tissue factor, such as a recombinant tissue factor having no membrane binding sequence or having only sequence of the extracellular domain.

According to an advantageous embodiment, the recombinant compound for generating thrombin is combined, preferably mixed, with one, preferably at least two different phospholipids.

Excellent results have been obtained, when using calcium phosphate containing particles with a mean size comprised between 150 μm and 500 μm.

For example, the calcium phosphate containing particles are hydroxyapatite particles with substantially no sharp and pointed edges. Such particles have been for example been submitted to a rolling treatment.

According to a detail of an advantageous embodiment, the weight ratio peptide of formula GTPGPQGIAGQRGVV/calcium phosphate containing particles is comprised between 0.00005 and 0.2, advantageously between 0.0001 and 0.1, preferably between 0.0002 and 0.05.

While various fibrinogen containing solution can be used (such as PPP, etc.), a platelet rich plasma is preferably used. Such a platelet rich plasma has advantageously a platelet concentration of 1,500,000 to 2,000,000 platelets per microlitre.

According to a specific embodiment, the platelet rich plasma having a platelet concentration of 1,500,000 to 2,000,000 platelets per microlitre, while from 1 to 200 μg (advantageously between 2 and 100 μg, preferably between 5 and 75 μg) recombinant tissue factor in dry form per ml PRP, from 1 to 4,0001 μg (advantageously from 5 to 3,000 μg, preferably from 50 to 2,000 μg) of phospholipids, from 0.01 to 10 g (advantageously from 0.5 to 5 g, preferably less than about 3 g) of hydroxyapatite particles per ml PRP and from 1 to 5,000 ng (advantageously from 5 to 1,500 ng, preferably from 10 to 1,000 ng) peptide of formula GTPGPQGIAGQRGVV per ml PRP are contacted with the platelet rich plasma.

The weight ratio peptide of formula GTPGPQGIAGQRGVV/hydroxyapatite particles is comprised between 0.0001 and 0.001, advantageously between 0.00015 and 0.0005, preferably about 0.00025.

The invention relates also to a kit for the preparation of a bone generating composition prepared by contacting a fibrinogen containing solution with a recombinant compound for generating thrombin in presence of at least a phospholipid, and inorganic particles containing calcium phosphate and having a mean particle size lower than 750 μm, and preferably also with a synthetic amino acid peptide of formula GTPGPQGIAGQRGVV, said kit comprising at least:

at least one system selected from the group consisting of a vial containing a recombinant compound for generating thrombin (possibly the synthetic amino acid peptide of formula GTPGPQGIAGQRGVV) and inorganic particles containing calcium phosphate and having a mean particle size lower than 750 μm, two distinct vials, a first containing a recombinant compound for generating thrombin, while the second contains the possible synthetic amino acid peptide of formula GTPGPQGIAGQRGVV, inorganic particles containing calcium phosphate and having a mean particle size lower than 750 μm.

Most preferably, at least one vial contains at least a buffer agent and at least an antibiotic.

Preferably, the inorganic particles containing calcium phosphate and having a mean particle size lower than 750 μm are hydroxyapatite particles with substantially no sharp and pointed edges.

Advantageously, the recombinant compound for generating thrombin is a recombinant tissue factor with phospholipids.

Advantageously, the recombinant compound for generating thrombin is a recombinant tissue factor having no membrane binding sequence or having only sequence of the intracellular domain.

According to an advantageous embodiment, the recombinant compound for generating thrombin is combined, preferably mixed, with one, preferably at least two different phospholipids.

According to a detail of specific embodiment, the calcium phosphate containing particles have a mean size comprised between 150 μm and 500 μm.

According to a preferred embodiment of the kit, the weight ratio peptide of formula GTPGPQGIAGQRGVV/calcium phosphate containing particles is comprised between 0.0001 and 0.001, advantageously between 0.00015 and 0.0005, preferably about 0.00025.

Advantageously, the first vial is adapted for containing PRP from the patient, while the system contains a recombinant compound for generating thrombin in a dry form, the peptide in dry form and the calcium phosphate inorganic particles in dry form.

According to a detail of a preferred method of the invention, the coagulation of the platelet rich plasma is carried out in presence of at least one antibiotic and/or at least one antibiotic is added to the mixture after the coagulation of the platelet rich plasma. The antibiotic or mixture of antibiotics can possibly be added to the bone particles and/or to the bone before its grinding and/or to the recombinant compound that generates thrombin and/or to a phospholipid. Preferably, an antibiotic or a mixture of antibiotics is mixed with the recombinant compound for generating thrombin (recombinant compound that generates thrombin), preferably with a recombinant tissue factor or a recombinant thromboplastin.

According to a specific embodiment, the kit comprises advantageously at least an antibiotic formulation selected from the group consisting of oral antibiotic formulation, injectable antibiotic formulation, topic antibiotic formulation, spray antibiotic formulation and inhaled antibiotic formulation, said formulation being suitable for administering to the patient an efficient dose of antibiotic at the place where bone has to be generated. Oral formulation and injectable formulation are preferred.

According to a detail of a preferred embodiment, the amount of antibiotic or antibiotics added to the bone generating product or used during the coagulation of the platelet rich plasma bone generating product or administered prior, during and/or after the application of the bone generating product to the patient is such that the bone generating product contains from 0.001 to 10% by weight antibiotic or antibiotics (calculated in its dry forms), advantageously from 0.01% to 5% by weight, preferably from 0.02 to 1%, for example from 0.05% to 0.4% by weight, more specifically from 0.2 to 0.3%. The antibiotic is advantageously selected from the group consisting of antibiotics having an anti osteoclasts effect (more specifically antibiotics of the tetracyclin group, Vibrarnycin®, Doxycycline®, Minocycline, Minocin® (Wyeth-Lederlee)), mixtures of antibiotics having an anti osteoclasts effect, and mixtures of one or more antibiotics having an anti osteoclasts effect with one or more other antibiotic(s) (preferably macrolide, penicillin based compounds, etc. and mixtures thereof).

Most preferably, at least one antibiotic is added to the mixture containing at least platelet rich plasma and calcium phosphate containing compound, before adding the recombinant compound that generates thrombin, but advantageously when adding the recombinant compound that generates thrombin.

When no antibiotic is added to the mixture or when a low amount of antibiotic is added to the mixture, antibiotic(s) are advantageously given to the patient by oral administration, by injection, by topic application, by inhalation, preferably by oral administration and/or by injection (most preferably injection in the blood or percutaneous injection), prior and/or during and/or after the application of the bone generating product to the patient.

The invention relates also to the use of a recombinant compound for generating thrombin in mixture with at least one phospholipid for the preparation of a bone generating product from a mixture of a platelet rich plasma and an effective amount of calcium containing compound for inducing bone generation.

A further object of the invention is a mixture containing a recombinant compound for generating thrombin, at least one phospholipid, and a calcium containing compound, the weight ratio calcium from the calcium containing compound/recombinant compound for generating thrombin being greater than 0.5, advantageously greater than 2.

The said has advantageously the form of a dry powder.

The calcium containing compound is preferably selected from the group consisting of calcium containing salts, β-tricalcium phosphate, particles of denatured bone and mixtures thereof.

Still a further object of the invention is a mixture containing a recombinant compound for generating thrombin, at least one phospholipid and at least one antibiotic. The weight ratio antibiotic(s) (as dry matter) present in the mixture/recombinant compound for generating thrombin (as dry matter) present in the mixture is advantageously greater than 1:1, preferably greater than 3:1, most preferably greater than 5:1 and more specifically greater than 10:1. For example the said ratio is comprised between 5:1 and 50:1, more specifically between 10:1 and 25:1.

The antibiotic is advantageously selected from the group consisting of antibiotics having an anti osteoclasts effect (more specifically antibiotics of the tetracyclin group, Vibramycin®, Doxycycline®, Minocycline, Minocin® (Wyeth-Lederlee)), mixtures of antibiotics having an anti osteoclasts effect, and mixtures of one or more antibiotics having an anti osteoclasts effect with one or more other antibiotic(s) (preferably macrolide, penicillin based compounds, etc. and mixtures thereof).

The recombinant compound that generates thrombin is advantageously a recombinant thromboplastin or a recombinant tissue factor.

Advantageously, the recombinant compound for generating thrombin is a recombinant tissue factor, such as a recombinant tissue factor having no membrane binding sequence or having only sequence of the extracellular domain.

According to an advantageous embodiment, the recombinant compound for generating thrombin is combined, preferably mixed, with one, preferably at least two different phospholipids.

The invention relates also to a method for the preparation of a sealant, in which a fibrinogen containing solution is contacted, preferably mixed, with a recombinant compound for generating thrombin in presence of at least a phospholipid. Advantageously, the recombinant compound for generating thrombin is a recombinant thromboplastin.

Advantageously, a gel is formed by contacting the fibrinogen containing solution with a recombinant compound for generating thrombin in presence of at least a phospholipid, at least an antibiotic and at least an effective amount of a buffer, so that the gel is formed at a pH kept between 6 and 8, advantageously between 7 and 7.5.

Preferably, a buffered solution containing the antibiotic(s) and the buffer agent(s) is prepared and contacted with the fibrinogen containing solution. The pH of said buffered solution is advantageously comprised between 6 and 8, most preferably between 7 and 7.5. The buffered solution may possibly, but advantageously, contain one or more recombinant compound for generating thrombin and/or one or more phospholipids.

Possible buffers are for example TRIS buffer, solution of Ringé, sodium bicarbonate, and mixture thereof.

Preferably, the fibrinogen containing solution is contacted with a recombinant compound for generating thrombin in presence of at least two different phospholipids.

Most preferably, the fibrinogen containing solution is contacted with a recombinant compound for generating thrombin in presence of at least one phospholipid selected from the group consisting of phosphatidylserine, derivatives thereof, phosphatidylserine having at least one fatty acid side chain, phosphatidylcholine, derivatives thereof, and phosphatidylcholine having at least one fatty acid side chain, lo preferably at least two phospholipids selected from said group.

The fatty acid side chain of phosphatidylcholine is advantageously selected from the group consisting of fatty acid chains with at least one double bond and with 6 to 24 carbon atoms, preferably with 16 to 18 carbon atoms.

Before the formation of the sealant, the mixed solutions have advantageously a pH substantially equal to the physiological pH, for example a pH comprised between 6.5 and 8, preferably about 7-7.5, pH measured at 37° C.

Advantageously, a platelet rich plasma is used as fibrinogen containing solution. The platelet rich plasma has advantageously a platelet concentration of 1,500,000 to 2,000,000 platelets per microlitre.

Preferably, the platelet rich plasma having a platelet concentration of 1,500,000 to 2,000,000 platelets per microlitre, while from 0.05 to 5 μg thromboplastin in dry form per microlitre and from 0.01 to 4 μg (advantageously from 0.1 to 0.4 μg, preferably from 0.2 to 0.3 μg) antibiotic(s) (as dry matter) per microlitre are contacted with the platelet rich plasma in presence of an effective amount of buffer agent(s) for regulating the pH between 6 and 8, advantageously between 7 and 7.5 during the gelling.

When using a platelet rich plasma, in order to avoid as much as possible complication and in order to improve as much as possible the graft of the sealant in a patient, the fibrinogen containing solution, advantageously the platelet containing solution, preferably the platelet rich plasma used is prepared from the plasma of the patient and/or from a histocompatible plasma (i.e. immunological histocompatibility).

The fibrinogen containing solution, preferably the platelet rich plasma, is advantageously contacted with at least a recombinant compound for generating thrombin in presence of at least one, preferably two different phospholipids, and in presence of at least an antibiotic.

Preferably, the fibrinogen containing solution, preferably the platelet rich plasma, is contacted with a solution containing at least a recombinant compound for generating thrombin and at least one, preferably two different phospholipids.

When using one or more antibiotics, the amount of antibiotic(s) used is advantageously such that the sealant contains from 0.001 to 10% by weight antibiotic or antibiotics, advantageously from 0.01% to 5% by weight, preferably from 0.02 to 1%, most preferably from 0.05 to 0.4%. The weight ratio antibiotic(s) (calculated as dry matter) present in the sealant mixture / recombinant compound for generating thrombin used in the sealant mixture is advantageously greater than 1:1, preferably greater than 3:1, most preferably greater than 5:1 and more specifically greater than 10:1. For example the said ratio is comprised between 5:1 and 50:1, more specifically between 10:1 and 25:1.

The antibiotic is advantageously selected from the group consisting of antibiotics having an anti osteoclasts effect (more specifically antibiotics of the tetracyclin group, Vibramycin®, Doxycycline®, minocycline, minocin® (Wyeth-Lederlee)), mixtures of antibiotics having an anti osteoclasts effect, and mixtures of one or more antibiotics having an anti osteoclasts effect with one or more other antibiotic(s) (preferably macrolide, penicillin based compounds, etc. and mixtures thereof).

The invention relates also a kit for the preparation of a sealant according to the invention, said kit comprising:

possibly a first vial adapted for containing a fibrinogen containing material, preferably a fibrinogen containing solution;

a second vial containing a recombinant compound for generating thrombin, preferably a solution containing said recombinant compound;

possibly, a third vial containing a solution to be added to the first vial and/or second vial for the preparation of a fibrinogen containing solution and/or a solution containing a recombinant compound for generating thrombin;

in which the first vial and/or the second vial and/or the third vial contains at least a phospholipid, preferably at least two different phospholipids, and in which the first vial and/or the second vial and/or the third vial (preferably the second and/or the third vial) contains at least an antibiotic.

Advantageously, the first vial and/or the second vial and/or the third vial contains at least a buffer agent. Preferably, the second vial contains at least a phospholipid and at least an antibiotic, while the third vial contain the buffer agent(s).

Most preferably, the first vial contains fibrinogen containing material, while the second vial contains a recombinant compound for generating thrombin in a dry form.

The invention relates also to the use of a recombinant compound for generating thrombin in mixture with at least one phospholipid and at least one peptide of the formula GTPGPQGIAGQRGVV for the preparation of a bone generating product from a mixture of a platelet rich plasma and an effective amount of calcium containing compound for inducing bone generation.

The invention still further relates to a method for inducing bone generation to a patient in need. In said method, a bone generating product is applied at the place of the patient where bone has to be generated.

The method of the invention comprises the step of:

applying at the place where bone has to be generated a bone generating product comprising a coagulated matrix of platelet rich plasma with a recombinant compound for generating thrombin in presence of at least a phospholipid, a synthetic amino acid peptide of formula GTPGPQGIAGQRGVV, and inorganic particles containing calcium phosphate and having a mean particle size lower than 750 μm.

Advantageously, the calcium phosphate containing compound is selected from the group consisting of bone particles, mixture of bone particles with calcium containing additives, synthetic hydroxyapatite, and mixtures thereof.

Preferably, the recombinant compound for generating thrombin is a recombinant tissue factor or thromboplastin.

According to an embodiment, the bone generating product further comprises at least a buffer agent and an antibiotic.

Preferably, the calcium phosphate containing particles have a mean size comprised between 150 μm and 500 μm.

According to a preferred embodiment, the weight ratio peptide of formula GTPGPQGIAGQRGVV/calcium phosphate containing particles is comprised between 0.0001 and 0.001, advantageously between 0.00015 and 0.0005, preferably about 0.00025.

According to a detail of an embodiment of the method, the patient is treated with at least an antibiotic, prior and/or during and/or after the application of the bone generating product to the patient. According to a specific embodiment, the fibrinogen containing solution, advantageously the platelet containing solution, preferably the platelet rich plasma is prepared from the blood of a patient treated with an antibiotic, such as with an effective amount of antibiotic or from a histocompatible blood of a human treated with an antibiotic.

Preferably, the patient is submitted to at least one treatment with at least one antibiotic, said treatment being selected from the group consisting of:

oral administration of an efficient dose or effective amount of at least one antibiotic at least after the application of the bone generating product to the patient;

oral administration of an efficient dose or effective amount of at least one antibiotic at least prior the application of the bone generating product to the patient;

injection of an efficient dose or effective amount of at least one antibiotic at least after the application of the bone generating product to the patient;

injection of an efficient dose of at least one antibiotic at least prior the application of the bone generating product to the patient;

administration of an efficient dose or effective amount of at least one antibiotic at least for one day prior the application of the bone generating product and at least for one day after the application of the bone generating product to the patient.

According to a specific embodiment, an effective amount of antibiotic is administered (most preferably orally or by injection) to the patient before the application of the bone generating product of the invention, as well as after said application.

Details and characteristics of the product and the process of the invention will appear from the following description of examples.

### DESCRIPTION OF EXAMPLES

For the preparation of the said examples, the following products have been used:

PRP: platelet rich plasma of the patient to which a bone graft has to be placed. The platelet concentration of the plasma was 1,800,000 platelets per microliter of the plasma. The PRP was subjected to known usual treatments for the removal leucocyte, for obtaining a maximum proportion of living platelets, for bacteriological control, said PRP being active at least for 5 days. Prior its use, the PRP was shaken at a temperature of 37° C., the said shaking being achieved by shaking the container containing the PRP.

Thromboplastin: The thromboplastin used was a thromboplastin sold under the Trademark Innovin by the company DADE AG( Duidingen, Switzerland). The thromboplastin is a recombinant human tissue factor lyophilized combined with synthetic phospholipids, namely phosphatidylserine and phosphatidylcholine, said phospholipids having at least one fatty acid side chain, the fatty acid side chain being selected from the group consisting of fatty acid chains with at least one double bond and with 16-18 carbon atoms. Innovin is free of prothrombin, free of factor FVII, and free of factor FX. Calcium is present in Innovin. Innovin is a known product for diagnostic purposes. Innovin contains also some calcium, a zwitterionic buffer system at physiological pH.

Innovin thromboplastin comprises a mixture of tissue factor and phospholipids, with a weight ratio tissue factor/phospholipids of about 1/300. (molar ratio TF/phospholipids of about 1/10,000).

Other thromboplastin can also be used, such as thromboplastin sold by American Diagnostica or thromboplastin developed by Henogen SA. The thromboplastin developed by Henogen SA comprises a tissue factor without membrane binding sequence and without intracellular domain. The Henogen tissue factor comprises thus only extra cellular domain. The Henogen tissue factor was expressed by yeast and recovered as soluble and glycosylated form in the culture medium. The tissue factor was purified by chromatography (in one or two steps, for example in two steps). The thromboplastin formulation of Henogen comprises also phospholipids, phosphatidyl-serine and phosphatidylcholine with a weight ratio phosphatidyl-serine/phosphatidylcholine of 3:7. The molar ratio TF/phospholipids is about 1/10,000. The recombinanttissue factor is mixed with said phospholipids.

Bone particles: The bone particles have been prepared from iliac bones or craniofacial bone of the patient to whom a bone graft is needed. The fresh bone of the patient was ground in bone flakes (bone meal) having an average diameter of 1 mm. The bone particles are added to the PRP just after their preparation.

Water: water used is distilled, sterilized, pyrogen free water.

PepGen P-15®: this product sold by Friadent, Germany is a peptide enhanced by natural hydroxyapatite. The peptide has the formula GTPGPQGIAGQRGVV. Said peptide is bound to natural calcium phosphate particles (hydroxyapatite) with a size comprised between 250 μm and 420 μm. The particles are anorganic bone mineral heated at a temperature higher than 1100° C. The particles have been submitted to a rolling so as to remove sharp and pointed edges. The weight ratio peptide of formula GTPGPQGIAGQRGVV/calcium phosphate particle is about 250/1,000,000 or 0.00025. The particles are at least partly coated with said peptide.

PepGen P-15 Flow®: This product is similar to the product disclosed here above, except that it contains a resorbable gel matrix (made of sodium salt of a polycarboxymethyl ether of cellulose, glycerol and hydrogel).

### Example 1

In said example, 50 ml of PRP was placed in a sterilized container. A volume of 10 ml of bone particles (craniofacial origin) was added to the PRP and mixed. The recipient is then heated under sterile conditions at 37.5° C. (for example by using a water bath having a temperature of 37.5° C., the said bath containing water and 0.9% NaCl), in an oxygen containing atmosphere.

10 mg Innovin was mixed with 2 ml distilled, sterile and pyrogen free water. The mixture water+Innovin was added to the PRP+bone particles mixture kept at a temperature of 37.5° C.

After about 10 minutes, without stirring, a gel is formed in the recipient, said gel being a bone generating product suitable for implant to the patient.

### Example 2

Example 1 has been repeated, except that 20 mg Innovin was mixed with 2 ml distilled, sterile and pyrogen free water, and was added to the mixture PRP+bone particles.

### Examples 3 to 9

In said examples, example 1 was repeated except that the amount of reagents used was different.

| | | | | | | | |
|---|---|---|---|---|---|---|---|
| Example | 3 | 4 | 5 | 6 | 7 | 8 | 9 |

| | | | | | | | |
|---|---|---|---|---|---|---|---|
| PRP(ml) | 50 | 50 | 50 | 50 | 50 | 50 | 50 |
| Bone particles | 10 | 10 | 15 | 40 | 30 | 25 | 50 |
| (ml) | | | | | | | |
| craniofacial | | | | | | | |
| Innovin (mg) | 20 | 10 | 10 | 10 | 10 | 20 | 10 |
| Water (ml) | 2 | 2 | 2 | 2 | 2 | 4 | 4 |

### Examples 10 to 19

In said examples, example 1 was repeated except that the amount of reagents used was different.

| Example | 10 | 11 | 12 | 13 | 14 | 15 | 16 | 17 | 18 | 19 |
|---|---|---|---|---|---|---|---|---|---|---|
| PRP(ml) | 50 | 50 | 50 | 50 | 50 | 50 | 50 | 50 | 50 | 50 |
| Bone particles | | | | 10 | 20 | 10 | 10 | 10 | 20 | 10 |
| (ml) | | | | | | | | | | |
| craniofacial | | | | | | | | | | |
| Bone particles | 10 | 20 | 40 | 10 | 10 | 20 | 10 | 10 | 20 | 30 |
| (ml) iliac | | | | | | | | | | |
| Innovin(mg) | 20 | 20 | 10 | 20 | 10 | 10 | 20 | 30 | 10 | 10 |
| Water (ml) | 2 | 2 | 4 | 2 | 2 | 2 | 2 | 2 | 4 | 4 |

The bone generating product of said examples 1 to 19 having the form of a gel can easily be implanted in a patient, for example in a human patient suffering a major maxillofacial atrophy. The bone generating product of the invention can easily be compacted in recesses of bones, and can be easily be shaped.

The bone generating product of the invention was used for volunteers suffering a major maxillofacial atrophy. Sinus lift grafts and on lay graft on the maxillofacial bone have been carried out. These tests have show a bone growth or the generation of bone where the bone generating product of the invention was applied.

### Example 20

A human bone was denatured and γ-irradiated so as to be prion free. The bone was ground in particles having an average (by weight) of 0.2 mm. After drying, 10 g of bone particles were dry mixed with 10 mg dry INNOVIN, so as to obtain a mixture of recombinant compound for generating thrombin, phospholipid and a high level of calcium containing compound.

The so prepared mixture was then used for the preparation of a bone generating product of the invention.

The method of example 1 was repeated, except that the mixture 10 mg INNOVIN+10 g denatured bone particles was used instead of 10 mg INNOVIN alone, and except that a larger amount of sterile water was used (5-10 ml), amount water adjusted so as to prepare a pasta.

### Example 21

Example 1 was repeated, except that before adding the recombinant thromboplastin, 200 μg Vibramycin® per ml mixture of PRP and bone particles was added.

### Example 22

Example 1 was repeated, except that before adding the recombinant thromboplastin, 100 μg Minocycline (Minocin®)per ml mixture of PRP and bone particles was added.

### Example 23

Example 1 was repeated, except that before adding the recombinant thromboplastin, 50 μg Minocycline (Minocin®) per ml mixture of PRP and bone particles was added.

### Example 24

Example 1 was repeated, except that before adding the recombinant thromboplastin, 20 μg Minocycline (Minocin®) per ml mixture of PRP and bone particles was added.

### Example 25

10 mg of Innovin was mixed with 100 mg Vibramycin®. The presence of Vibramycin seems to improve the stability and efficiency of the Innovin.

The mixture Innovin—Vibramycin® was used as in example 1 for the preparation of the gel.

### Example 26

10 mg Innovin was mixed with 5 ml water. Thereafter, 100 mg Vibramycin was added to the said aqueous mixture of Innovin. The mixture was then lyophilized so as to obtain a powder or cake containing Innovin and Vibramycin.

The lyophilized product was then used as in example 25.

As in the preparation process of Innovin, an aqueous mixture of recombinant thromboplastin and phospholipid is lyophilized, it is possible to add the said antibiotic (for example Vibramycin ) to the aqueous mixture before its lyophilization (for example before and/or after the addition of Ca⁺⁺, buffer and stabilizers to the mixture as carried out in the preparation process of Innovin).

### Examples 27 to 29

Examples 24 to 26 were repeated except that Minocin® (Wyeth-Lederlee) was used instead of Vibramycin®.

### Examples 30 to 59

Examples 1 to 29 have been repeated except that the recombinant tissue factor 4500L/B of American diagnostica Inc. was used instead of Innovin. The recombinant tissue factor 4500L/B2 of American diagnostica Inc. can also be used.

### Example 60

A sealant was prepared by mixing 50 ml of PRP with 2 ml aqueous solution containing 10 mg Innovin, 100 mg Minocin® and a physiologically acceptable buffer (in an amount sufficient for having a pH of about 7.2 for the aqueous solution before its mixing with the PRP, for example a solution of sodium bicarbonate). A sealant gel was so obtained.

### Example 61

50 ml PRP was placed in a chamber of a first syringe, while 2 ml aqueous solution containing 10 mg Innovin, 100 mg Minocin® and a physiologically acceptable buffer (sodium bicarbonate) was placed in a chamber of a second syringe. The two syringes were connected to a mixer for mixing PRP with Innovin, Minocin and buffer before applying the mixture to the wound, and to a means for delivering to the mixer on a continuous manner 0.05 ml Innovin solution per ml of PRP.

### Example 62

Example 61 was repeated except that 10 ml of an aqueous solution containing 10 mg Innovin and 200 mg Vibramycin® and buffer agent (the pH of the solution being about 7.2) was used, and that the means delivers to the mixer on a continuous manner 0.2ml Innovin-Vibramycin solution per ml of PRP.

### Example 63

A kit for the preparation of a sealant, said kit comprising:

a first vial adapted for containing PRP, for example of the patient;

a second vial containing Innovin and Vibramycin in a dry form,

a third vial containing sterile water and a buffer (amount sufficient for ensuring a pH of 7.2, when mixing the three vials together, preferably the second and third vials are first mixed together and the mixture thereof is mixed with the content of the first vial) for reconstituting the solution containing Innovin-Vibramycin.

Advantageously, the kit further comprises means for mixing the PRP with the reconstituted Innovin-Vibramycin solution and means for applying the mixture on the wound, for example by spraying.

### Example 64

Sealant composition of the invention and pasta (bone generating product), especially as prepared in the previous example, were used for coating an artificial bone, for example a bone having been submitted to one or more sterilization treatments.

### Example 65

In said example, 50 ml of PRP was placed in a sterilized container. A volume of 50 ml of PepGen P-15 was added to the PRP and mixed. The recipient is then heated under sterile conditions at 37.5° C. (for example by using a water bath having a temperature of 37.5° C., the said bath containing water and 0.9% NaCl), in an oxygen containing atmosphere.

10 mg Innovin was mixed with 2 ml distilled, sterile and pyrogen free water. The mixture water+Innovin was added to the PRP+PepGen P-15 mixture kept at a temperature of 37.5° C.

After about 10 minutes, without stirring, a gel is formed in the recipient, said gel being a bone generating product suitable for implant to the patient.

### Example 66

Example 65 has been repeated, except that 20 mg Innovin was mixed with 2 ml distilled, sterile and pyrogen free water, and was added to the mixture PRP+PepGen P-15.

### Examples 67 to 73

In said examples, example 65 was repeated except that the amount of reagents used was different.

| Example | 67 | 68 | 69 | 70 | 71 | 72 | 73 |
|---|---|---|---|---|---|---|---|
| PRP(ml) | 50 | 50 | 50 | 50 | 50 | 50 | 50 |
| PepGen P-15 | 35 | 35 | 45 | 40 | 60 | 75 | 75 |
| (ml) | | | | | | | |
| Innovin (mg) | 20 | 10 | 5 | 10 | 10 | 20 | 10 |
| Water (ml) | 2 | 2 | 2 | 2 | 2 | 4 | 4 |

### Examples 74 to 83

In said examples, example 65 was repeated except that the amount of reagents used was different.

| Example | 74 | 75 | 76 | 77 | 78 | 79 | 80 | 81 | 82 | 83 |
|---|---|---|---|---|---|---|---|---|---|---|
| PRP(ml) | 50 | 50 | 50 | 50 | 50 | 50 | 50 | 50 | 50 | 50 |
| Bone particles | | | | 10 | 20 | 10 | 10 | 10 | 20 | 10 |
| (ml) | | | | | | | | | | |
| craniofacial | | | | | | | | | | |
| Bone particles | 10 | 20 | 40 | 10 | 10 | 20 | 10 | 10 | 20 | 30 |
| (ml) iliac | | | | | | | | | | |
| Innovin(mg) | 20 | 20 | 10 | 20 | 10 | 10 | 20 | 30 | 10 | 10 |
| PepGen P-15 | 35 | 40 | 50 | 50 | 50 | 60 | 65 | 70 | 75 | 75 |
| (ml) | | | | | | | | | | |
| Water (ml) | 2 | 2 | 4 | 2 | 2 | 2 | 2 | 2 | 4 | 4 |

The bone generating product of said examples 65 to 83 having the form of a gel can easily be implanted in a patient, for example in a human patient suffering a major maxillofacial atrophy. The bone generating product of the invention can easily be compacted in recesses of bones, and can be easily be shaped.

The bone generating product of the invention was used for volunteers suffering a major maxillofacial atrophy. Sinus lift grafts and on lay graft on the maxillofacial bone have been carried out. These tests have show a bone growth or the generation of bone where the bone generating product of the invention was applied.

### Example 84

A human bone was denatured and γ-irradiated so as to be prion free. The bone was ground in particles having an average (by weight) of 0.2 mm. After drying, 10 g of bone particles were dry mixed with 10 mg dry Innovin, so as to obtain a mixture of recombinant compound for generating thrombin, phospholipid and a high level of calcium containing compound.

The so prepared mixture was then used for the preparation of a bone generating product of the invention.

The method of example 65 was repeated, except that the mixture 10 mg Innovin+10 g denatured bone particles was used instead of 10 mg Innovin alone, and except that a larger amount of sterile water was used (5-10 ml), amount water adjusted so as to prepare a paste.

### Example 85

Example 1 was repeated, except that before adding the recombinant thromboplastin, 200 μg Vibramycin® per ml mixture of PRP was added.

### Example 86

### Example 65 was repeated, except that before adding the recombinant thromboplastin, 100 μg Minocycline (Minocin®) per ml mixture of PRP was added.

### Example 87

### Example 65 was repeated, except that before adding the recombinant thromboplastin, 50 μg Minocycline (Minocin®) per ml mixture of PRP was added.

### Example 88

Example 1 was repeated, except that before adding the recombinant thromboplastin, 20 μg Minocycline (Ninocin®) per ml mixture of PRP was added.

### Examples 89 to 112

### Examples 65 to 88 have been repeated, except that PepGen P-15 Flow was used instead of PepGen P-15.

### Examples 113 to 136

Examples 65 to 88 have been repeated except that Innovin was replaced by another recombinant tissue factor mixed with phospholipids. For example, the recombinant tissue factor is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof. Preferably, the recombinant compound for generating thrombin is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof, the fatty acid side chain being selected from the group consisting of fatty acid chains with at least one double bond and with 6 to 24 carbon atoms, most preferably with 16 to 18 carbon atoms.

According to a most preferred embodiment, the recombinant compound for generating thrombin is combined with a mixture of at least two phospholipids, a first phospholipid being selected from the group consisting of phosphatidylserine, derivatives thereof, phosphatidylserine having at least one fatty acid side chain, and mixtures therof, the fatty acid side chain of the phosphatidylserine having at least one fatty acid side chain being selected from the group consisting of fatty acid chains with at least one double bond and with 6 to 24 carbon atoms, preferably 16 to 18, while the second phospholipid is selected from the group consisting of phosphatidylcholine, derivatives thereof, phosphatidylcholine having at least one fatty acid side chain, and mixtures thereof, the fatty acid side chain of the phosphatidycholine having at least one fatty acid side chain being selected from the group consisting of fatty acid chains with at least one double bond and with 6 to 24 carbon atoms.

The weight ratio recombinant tissue factor/phospholipids is comprised for example between 1:500 and 1:50, such as in this example between 1:300 and 1:200, i.e. about 1:250.

In said examples, Innovin was replaced at the rate of 1.5 mg recombinant tissue factor+phospholipids per 10 mg Innovin used in examples 65 to 88.

Sealant were also prepared by mixing PRP, recombinant tissue factor of Henogen SA and phospholipids, with and without antibiotics.

What we claim is:

1. A method for the preparation of a sealant, in which a fibrinogen containing solution is contacted with a recombinant compound for generating thrombin in presence of at least a phospholipid, at least a buffer and at least an antibiotic.

2. The method of claim 1, in which a gel is formed by contacting the fibrinogen containing solution with a recombinant compound for generating thrombin in presence of at least a phospholipid, at least an antibiotic and at least a effective amount of buffer, so that the pH of the contacted fibrinogen solution is kept between 6 and 8, advantageously between 7 and 7.5 during the formation of the gel.

3. The method of claim 2, in which a buffered solution containing the antibiotic(s) and buffer agent(s) is prepared, said buffered solution having a pH comprised between 6 and 8, and in which the fibrinogen containing solution is contacted with said buffered solution.

4. The method of claim 1, in which the recombinant compound for generating thrombin comprises a recombinant tissue factor.

5. The method of claim 1, in which the recombinant compound for generating thrombin is a recombinant tissue factor having no membrane binding sequence.

6. The method of claim 1, in which the fibrinogen containing solution is contacted with a recombinant compound for generating thrombin in presence of at least two different phospholipids.

7. The method of claim 1, in which the fibrinogen containing solution is contacted with a recombinant compound for generating thrombin in presence of at least one phospholipid selected from the group consisting of phosphatidylserine, derivatives thereof, phosphatidylcholine, and derivatives thereof.

8. The method of claim 1, in which the fibrinogen containing solution is contacted with a recombinant compound for generating thrombin in presence of at least two different phospholipids selected from the group consisting of phosphatidylserine, phosphatidylserine having at least one fatty acid side chain, phosphatidylcholine, and phosphatidylcholine having at least one fatty acid side chain.

9. The method of claim 1, in which the fibrinogen containing solution is contacted with a recombinant compound for generating thrombin in presence of at least one phosphatidylcholine having at least one fatty acid side chain, said fatty acid side chain being selected from the group consisting of fatty acid chains with at least one double bond and with 6 to 24 carbon atoms, preferably with 16 to 18 carbon atoms.

10. The method of claim 1, in which a platelet rich plasma is used as fibrinogen containing solution, said platelet rich plasma being contacted with a recombinant compound for generating thrombin in presence of at least a phospholipid and a buffer agent.

11. The method of claim 11, in which the platelet rich plasma has a platelet concentration of 1,500,000 to 2,000,000 platelets per microlitre.

12. A method for the preparation of a sealant, in which a fibrinogen containing solution is contacted with a recombinant tissue factor having no membrane binding sequence.

13. A method for the preparation of a sealant, in which a platelet rich plasma PRP having a platelet concentration of 1,500,000 to 2,000,000 platelets per microlitre is contacted with 1 to 200 μg recombinant tissue factor per ml PRP, in presence of 50 to 2000 μg phospholipids per ml PRP , of 10 to 400 μg antibiotic per ml PRP, and in presence of an effective amount of buffer agent for regulating the pH between 6 and 8 during the gelling.

14. A kit for the preparation of a sealant by contacting a fibrinogen containing solution with a recombinant compound for generating thrombin in presence of at least a phospholipid, at least a buffer and at least an antibiotic, said kit comprising at least:
one system selected from the group consisting of a vial containing a recombinant compound for generating thrombin, at least two different phospholipids, a buffer and an antibiotic; two distinct vials, a first containing a recombinant compound for generating thrombin and at least two different phospholipids, while the second contains a buffer and an antibiotic; three distinct vials, a first containing a recombinant compound for generating thrombin and at least two different phospholipids, the second containing a buffer and the third containing an antibiotic; and a system consisting of a vial system containing a recombinant compound for generating thrombin, at least two different phospholipids and a buffer, and an antibiotic formulation for oral administration.

15. The kit of claim 14, in which the recombinant compound for generating thrombin and the phospholipids are in dry form.

16. The kit of claim 14, in which the buffer and the antibiotic are in dry form.

17. A method for sealing at least a bleeding part of an living organism, in which a sealant film is formed on said bleeding part, by depositing on said bleeding part a composition prepared by mixing a fibrinogen containing solution, a recombinant compound for generating thrombin, at least two different phospholipids, a buffer and an antibiotic.

18. A method for sealing at least a bleeding part of an living organism, in which a sealant film is formed on said bleeding part, by depositing on said bleeding part a composition prepared by mixing a fibrinogen containing solution, a recombinant compound for generating thrombin, at least two different phospholipids, and a buffer, while an antibiotic is administered to the living organism before the sealing of the bleeding part.

19. A method for sealing at least a bleeding part of an living organism, in which a sealant film is formed on said bleeding part, by depositing on said bleeding part a composition prepared by mixing a fibrinogen containing solution, a recombinant compound for generating thrombin, at least two different phospholipids, and a buffer, and in which an antibiotic is administered to the living organism less than 12 hours after the sealing of bleeding part.

20. Bone generating product comprising a coagulated matrix of platelet plasma with a recombinant compound for generating thrombin in presence of at least one phospholipid, and an effective amount of calcium containing compound dispersed in the said matrix for inducing the formation of bone.

21. The bone generating product of claim 20, in which the calcium containing compound comprises bones particles.

22. The bone generating product of claim 20, in which the calcium containing compound comprises hydroxyapatite.

23. The bone generating product of claim 20, in which the recombinant compound for generating thrombin comprises a recombinant tissue factor.

24. The bone generating product of claim 20, said product comprising a coagulated matrix of platelet rich plasma with a recombinant compound for generating thrombin in presence of at least one phospholipid, and an effective amount of calcium containing compound dispersed in the said matrix for inducing the formation of bone.

25. The bone generating product of claim 20, which further comprises at least two different phospholipids.

26. The bone generating product of claim 20, in which the recombinant compound for generating thrombin is combined with phospholipids.

27. The bone generating product of claim 20, in which the recombinant compound for generating thrombin is combined with at least a phospholipid selected from the group consisting of phosphatidylserine, derivatives thereof, phosphatidylcholine, derivatives thereof, and mixtures thereof.

28. The bone generating product of claim 20, in which the recombinant compound for generating thrombin is combined with at least a phospholipid selected from the group consisting of phosphatidylserine, phosphatidylserine having at least one fatty acid side chain, phosphatidylcholine, phosphatidylcholine having at least one fatty acid side chain, and mixtures thereof, the fatty acid side chain being selected from the group consisting of fatty acid chains with at least one double bond and with 6 to 24 carbon atoms.

29. The bone generating product of claim 20, in which the recombinant compound for generating thrombin is combined with at least a phospholipid selected from the group consisting of phosphatidylserine, phosphatidylserine having at least one fatty acid side chain, phosphatidylcholine, phosphatidylcholine having at least one fatty acid side chain, and mixtures thereof, the fatty acid side chain being selected from the group consisting of fatty acid chains with at least one double bond and with 16 to 18 carbon atoms.

30. The bone generating product of claim 20, in which the recombinant compound for generating thrombin is combined with a mixture of at least two phospholipids, a first phospholipid being selected from the group consisting of phosphatidylserine, phosphatidylserine having at least one fatty acid side chain, and mixtures thereof, the fatty acid side chain of the phosphatidylserine having at least one fatty acid side chain being selected from the group consisting of fatty acid chains with at least one double bond and with 6 to 24 carbon atoms, while the second phospholipid is selected from the group consisting of phosphatidylcholine, phosphatidylcholine having at least one fatty acid side chain, and mixtures thereof, the fatty acid side chain of the phosphatidycholine having at least one fatty acid side chain being selected from the group consisting of fatty acid chains with at least one double bond and with 6 to 24 carbon atoms.

31. The bone generating product of claim 20, in which the thrombin generating product is a recombinant tissue factor.

32. The bone generating product of claim 31, in which the tissue factor is a tissue factor with no membrane binding sequence.

33. The bone generating product of claim 20, in which the calcium containing compound comprises bone particles selected from the group consisting of craniofacial bone particles, iliac bone particles and mixtures thereof.

34. The bone generating product of claim 33, in which the bone particles are particles of non denatured bones.

35. The bone generating product of claim 20, in which the bone particles have an average particle size comprised between 0.5 mm and 5 mm.

36. The bone generating product of claim 20, in which the calcium containing compound is selected from the group consisting of bone particles, mixture of bone particles with calcium containing additives, the product comprising from 15% to 50% by volume of bone particles.

37. The bone generating product of claim 20, in which the coagulated matrix is a coagulated matrix of platelet rich plasma having a platelet concentration of 1,500,000 to 2,000,000 platelets per microlitre of the matrix forming agents.

38. The bone generating product of claim 20, in which the coagulated matrix is a coagulated matrix of platelet rich plasma having a platelet concentration of 1,500,000 to 2,000,000 platelets per microlitre of the matrix forming agents and 0.05 to 5 μg thromboplastin in dry form per microlitre of the matrix forming agents.

39. The bone generating product of claim 20 for a patient, in which the platelet rich plasma is prepared from the plasma of the patient and in which the bone particles are prepared from a bone of the patient.

40. The bone generating product of claim 20, in which the coagulated matrix is associated with a bio compatible film.

41. The bone generating product of claim 20, which further contains at least an additive selected among the group consisting of growth factors, genes encoding growth factors, drugs, fatty acids, antibiotics, bactericides, virucides, compounds inducing the formation of matrixes, and mixtures thereof

42. The bone generating product of claim 41, which contains as antibiotic at least an antibiotic having an anti osteoclasts effect.

43. A bone generating product comprising a coagulated matrix of fibrinogen containing solution with a recombinant compound for generating thrombin in presence of at least a phospholipid, a synthetic amino acid peptide of formula GTPGPQGIAGQRGVV, and inorganic particles containing calcium phosphate and having a mean particle size lower than 750 μm.

44. The bone generating product of claim 43, in which the fibrinogen containing solution is a platelet containing plasma.

45. The bone generating product of claim 43, in which the coagulated matrix is a matrix of platelet rich plasma.

46. The bone generating product of claim 43, in which the calcium phosphate containing compound is selected from the group consisting of bone particles, synthetic hydroxyapatite, and mixtures thereof.

47. The bone generating product of claim 43, in which the recombinant compound for generating thrombin comprises a recombinant tissue factor.

48. The bone generating product of claim 43, which further comprises at least a buffer agent and an antibiotic.

49. The bone generating product of claim 43, in which the calcium phosphate containing particles have a mean size comprised between 150 μm and 500 μm.

50. The bone generating product of claim 43, in which the weight ratio peptide of formula GTPGPQGIAGQRGVV/calcium phosphate containing particles is comprised between 0.00005 and 0.2.

51. The bone generating product of claim 43, in which the calcium containing compound comprises bone particles selected from the group consisting of craniofacial bone particles, iliac bone particles and mixtures thereof.

52. The bone generating product of claim 50, in which the bone particles are particles derived from non-denatured bones.

53. The bone generating product of claim 43, in which the calcium phosphate containing particles have substantially no sharp and pointed edge.

54. The bone generating product of claim 43, in which the coagulated matrix is a coagulated matrix of platelet rich plasma having a platelet concentration of 1,500,000 to 2,000,000 platelets per microlitre of the matrix forming agents.

55. The bone generating product of claim 43, in which the thrombin generating product is a recombinant tissue factor.

56. The bone generating product of claim 54, in which the recombinant tissue factor is a tissue factor with no membrane binding sequence.

57. The bone generating product of claim 43 for a patient, in which the platelet rich plasma is prepared from the plasma of the patient and in which the bone particles are prepared from a bone of the patient.

58. The bone generating product of claim 43, in which the coagulated matrix is associated with a bio compatible film.

59. The bone generating product of claim 43, which further contains at least an additive selected among the group consisting of growth factors, genes encoding growth factors, drugs, fatty acids, bactericides, virucides, compounds inducing the formation of matrixes, and mixtures thereof

60. The bone generating product of claim 59, which contains at least an antibiotic having an anti osteoclasts effect.

61. A bone generating product comprising a coagulated matrix of platelet rich plasma prepared by gelling a mixture of platelet rich plasma with a platelet concentation of 1,500,000 to 2,000,000 platelets per microlite, from 1 to 200 μg recombinant tissue factor in dry form per ml platelet rich plasma, from 5 to 3,000 μg phospholipids per ml platelet rich plasma, from 0.01 to 5 g of hydroxyapatite particles with a mean particle size lower than 750 μm per ml platelet rich plasma and from 1 to 5,000 ng peptide of formula GTPGPQGIAGQRGVV per ml platelet rich plasma.

62. A method for the preparation of a bone generating product comprising a coagulated matrix of fibrinogen containing solution with a recombinant compound; for generating thrombin, and calcium containing particles dispersed in the said matrix, in which
a substantially homogeneous mixture is prepared by mixing fibrinogen containing solution with an amount of calcium containing compound effective for inducing the generation of bone when adding a recombinant thrombin generating compound and a phospholipid,
a recombinant thrombin generating compound and a phospholipid are added and mixed to the mixture prepared from the fibrinogen containing solution, and
the mixture recombinant thrombin generating compound, phospholipid, platelet rich plasma and calcium containing compound is kept under conditions for ensuring a coagulation of the fibrinogen containing solution and the formation of a matrix.

63. The method of claim 62, in which the fibrinogen containing solution is a platelet containing plasma.

64. The method of claim 62, in which the fibrinogen containing solution is a platelet rich plasma.

65. The method of claim 62, in which the calcium containing compound comprises bone particles.

66. The method of claim 62, in which the calcium containing compound comprises synthetic calcium phosphate particles.

67. The method of claim 62, in which the coagulation is carried out in presence of oxygen and substantially without stirring.

68. The method of claim 62, in which the recombinant compound for generating thrombin used for the coagulation comprises a recombinant tissue factor.

69. The method of claim 62, in which at least one phospholipid is added to a mixture selected from the group consisting of recombinant tissue factor, mixture of recombinant thrombin generating compound, platelet containing plasma and calcium containing compound, and mixture of platelet containing plasma and calcium containing compound.

70. The method of claim 62, in which the recombinant compound for generating thrombin is combined with phospholipids.

71. The method of claim 62, in which the recombinant compound for generating thrombin is combined with at least a phospholipid selected from the group consisting of phosphatidylserine, derivatives thereof, phosphatidylcholine, derivatives thereof, and mixtures thereof.

72. The method of claim 62, in which the recombinant compound for generating thrombin is combined with at least one phospholipid selected from the group consisting of phosphatidylserine, phosphatidylserine having at least one fatty acid side chain, phosphatidylcholine, phosphatidylcholine having at least one fatty acid side chain, and mixtures thereof, the fatty acid side chain being selected from the group consisting of fatty acid chains with at least one double bond and with 6 to 24 carbon atoms.

73. The method of claim 62, in which the recombinant compound for generating thrombin is combined with at least one phospholipid selected from the group consisting of phosphatidylserine, phosphatidylserine having at least one fatty acid side chain, phosphatidylcholine, phosphatidylcholine having at least one fatty acid side chain, and mixtures thereof, the fatty acid side chain being selected from the group consisting of fatty acid chains with at least one double bond and with 16 to 18 carbon atoms.

74. The method of claim 62, in which the recombinant compound for generating thrombin is combined with a mixture of at least two phospholipids, a first phospholipid being selected from the group consisting of phosphatidylserine, phosphatidylserine having at least one fatty acid side chain, and mixtures thereof, the fatty acid side chain of the phosphatidylserine having at least one fatty acid side chain being selected from the group consisting of fatty acid chains with at least one double bond and with 6 to 24 carbon atoms, while the second phospholipid is selected from the group consisting of phosphatidylcholine, phosphatidylcholine having at least one fatty acid side chain, and mixtures thereof, the fatty acid side chain of the phosphatidycholine having at least one fatty acid side chain being selected from the group consisting of fatty acid chains with at least one double bond and with 6 to 24 carbon atoms.

75. The method of claim 62, in which the calcium containing compound comprises bone particles selected from the group consisting of craniofacial bone particles, iliac bone particles and mixture thereof.

76. The method of claim 75, in which the bone particles are particles of non denatured bones.

77. The method of claim 62, in which the calcium containing compound comprises bone particles with an average particle size comprised between 0.5 mm and 5 mm.

78. The method of claim 62, in which the calcium containing compound comprises bone particles, and in which the amount of bone particles added to the fibrinogen containing solution corresponds to about 15% to 50% by volume of the mixture fibrinogen containing solution and bone particles.

79. The method of claim 62, in which the fibrinogen containing solution is a platelet containing plasma with a platelet concentration of 1,500,000 to 2,000,000 platelets per microlitre of the matrix forming agents.

80. The method of claim 61, in which the mixture platelet containing plasma, calcium containing compound and recombinant thrombin generating compound has a platelet concentration of 1,500,000 to 2,000,000 platelets per microlitre of the mixture without calcium containing compound and contains 0.05 to 5 μg thromboplastin in dry form per microlitre of the mixture without calcium containing compound.

81. The method of claim 62, for the preparation of a bone generating product for a patient, in which a platelet containing plasma is used as fibrinogen containing solution, the platelet containing plasma being selected from the group consisting of the plasma of the patient, a plasma histocompatible with the patient and mixtures thereof, and in which the calcium containing compound consists essentially of bone particles prepared from at least a bone selected from the group consisting of bones of the patient, bones histocompatible with the patient and mixtures thereof.

82. The method of claim 62, in which the thrombin generating product is a recombinant tissue factor.

83. The method of claim 82, in which the recombinant tissue factor is a recombinant tissue factor with no membrane binding sequence.

84. A method for the preparation of a bone generating composition, in which a fibrinogen containing solution is contacted with a recombinant compound for generating thrombin in presence of at least a phospholipid, a synthetic amino acid peptide of formula GTPGPQGIAGQRGVV, and inorganic particles containing calcium phosphate and having a mean particle size lower than 750 μm.

85. The method of claim 84, in which a gel is formed by contacting the fibrinogen containing solution with a recombinant compound for generating thrombin in presence of at least a phospholipid, a synthetic amino acid peptide of formula GTPGPQGIAGQRGVV, and said inorganic particles, whereby during the gel formation, the pH of the contacted fibrinogen solution is kept between 6 and 8.

86. The method of claim 84, in which the gel is formed in presence of an antibiotic and at least a buffer agent.

87. The method of claim 84, in which the recombinant compound for generating thrombin is a recombinant thromboplastin.

88. The method of claim 84, in which the calcium phosphate containing particles have a mean size comprised between 150 μm and 500 μm.

89. The method of claim 84, in which the calcium phosphate containing particles are hydroxyapatite particles with substantially no sharp and pointed edges.

90. The method of claim 84, in which the weight ratio peptide of formula GTPGPQGIAGQRGVV/calcium phosphate containing particles is comprised between 0.00005 and 0.2.

91. The method of claim 84, in which the fibrinogen containing solution is a platelet rich plasma.

92. The method of claim 91, in which the platelet rich plasma has a platelet concentration of 1,500,000 to 2,000,000 platelets per microlitre.

93. The method of claim 91, in which the platelet rich plasma PRP has a platelet concentration of 1,500,000 to 2,000,000 platelets per microlitre, while from 1 to 200 μg recombinant tissue factor in dry form per milliliter PRP, from 1 to 4000 μg phospholipids per ml PRP, from 0.01 to 10 g of hydroxyapatite particles per ml PRP and from 1 to 5,000 ng peptide of formula GTPGPQGIAGQRGVV per ml PRP are contacted with the platelet rich plasma.

94. The method of claim 84, in which the thrombin generating product is a recombinant tissue factor.

95. The method of claim 84, in which the recombinant tissue factor is a recombinant tissue factor with no membrane binding sequence.

96. Use of a recombinant compound for generating thrombin in mixture with at least one phospholipid for the preparation of a bone generating product from a mixture of a fibrinogen containing solution and an effective amount of calcium containing compound for inducing bone generation.

97. Mixture containing a recombinant compound for generating thrombin, at least one phospholipid, and a calcium containing compound, the weight ratio calcium from the calcium containing compound/recombinant compound for generating thrombin being greater than 0.5.

98. The mixture of claim 97, in which the weight ratio calcium from the calcium containing compound/recombinant compound for generating thrombin is greater than 2.

99. The mixture of claim 97, said mixture having the form of a dry powder.

100. The mixture of claim 97, in which the calcium containing compound is selected from the group consisting of calcium containing salts, particles of denatured bone and mixtures thereof.

101. Mixture containing a recombinant compound for generating thrombin, at least one phospholipid, and at least one antibiotic, the weight ratio antibiotic/recombinant compound for generating thrombin being greater than 1.

102. The mixture of claim 101, in which the antibiotic is an antibiotic having an anti osteoclasts effect.

103. The mixture of claim 101, in which the weight ratio antibiotic/recombinant compound for generating thrombin is greater than 5.

104. A kit for the preparation of a bone generating composition prepared by contacting a fibrinogen containing solution with a recombinant compound for generating thrombin in presence of at least a phospholipid, and inorganic particles containing calcium phosphate and having a mean particle size lower than 750 μm, said kit comprising at least:
at least one system selected from the group consisting of a vial containing a recombinant compound for generating thrombin, and inorganic particles containing calcium phosphate and having a mean particle size lower than 750 μm, two distinct vials, a first containing a recombinant compound for generating thrombin, while the second contains the inorganic particles containing calcium phosphate and having a mean particle size lower than 750 μm.

105. A kit for the preparation of a bone generating composition prepared by contacting a fibrinogen containing solution with a recombinant compound for generating thrombin in presence of at least a phospholipid, a synthetic amino acid peptide of formula GTPGPQGIAGQRGVV, and inorganic particles containing calcium phosphate and having a mean particle size lower than 750 μm, said kit comprising at least:
at least one system selected from the group consisting of a vial containing a recombinant compound for generating thrombin, the synthetic amino acid peptide of formula GTPGPQGIAGQRGVV and inorganic particles containing calcium phosphate and having a mean particle size lower than 750 μm, two distinct vials, a first containing a recombinant compound for generating thrombin, while the second contains the synthetic amino acid peptide of formula GTPGPQGIAGQRGVV and inorganic particles containing calcium phosphate and having a mean particle size lower than 750 μm.

106. The kit of claim 105, in which at least one vial contains at least a buffer agent and at least an antibiotic.

107. The kit of claim 105, in which the inorganic particles containing calcium phosphate and having a mean particle size lower than 750 μm are hydroxyapatite particles with substantially no sharp and pointed edges.

108. The kit of claim 105, in which the recombinant compound for generating thrombin comprises a recombinant tissue factor.

109. The kit of claim 105, in which the calcium phosphate containing particles have a mean size comprised between 150 μm and 500 μm.

110. The kit of claim 105, in which the weight ratio peptide of formula GTPGPQGIAGQRGVV/calcium phosphate containing particles is comprised between 0.00005 and 0.2.

111. The kit of claim 105, in which the system contains a recombinant compound for generating thrombin in a dry form, the peptide in dry form and the calcium phosphate inorganic particles in dry form.

112. The kit of claim 105, said kit comprising at least an antibiotic formulation selected from the group consisting of oral antibiotic formulation, injectable antibiotic formulation, topic antibiotic formulation, spray antibiotic formulation and inhaled antibiotic formulation.

113. The kit of claim 105, in which the thrombin generating product is a recombinant tissue factor.

114. The kit of claim 105, in which the recombinant tissue factor is a recombinant tissue factor with no membrane binding sequence.

115. A method for generating bone to a patient in need, said method comprising the step of:
applying at the place where bone has to be generated a bone generating product comprising a coagulated matrix of fibrinogen containing solution with a recombinant compound for generating thrombin in presence of at least a phospholipid, and inorganic particles containing calcium phosphate and having a mean particle size lower than 750 μm.

116. The method of claim 115, in which the calcium phosphate containing compound is selected from the group consisting of bone particles, derivatives thereof, synthetic hydroxyapatite, and mixtures thereof.

117. The method of claim 115, in which the coagulated matrix is prepared in presence of a synthetic amino acid peptide of formula GTPGPQGIAGQRGVV.

118. The method of claim 115, in which the recombinant compound for generating thrombin is a recombinant tissue factor.

119. The method of claim 115, in which the coagulated matrix further comprises at least a buffer agent and an antibiotic.

120. The method of claim 115, in which the calcium phosphate containing particles have a mean size comprised between 150 μm and 500 μm.

121. The method of claim 1 17, in which the weight ratio peptide of formula GTPGPQGIAGQRGVV/calcium phosphate containing particles is comprised between 0.00005 and 0.2.

122. The method of claim 115, in which the coagulated matrix is prepared in presence of a synthetic amino acid peptide of formula GTPGPQGIAGQRGVV, said amino acid peptide coating the calcium phosphate containing particles.

123. The method of claim 115, in which the patient is treated with at least an antibiotic.

124. The method of claim 115, in which the patient is submitted to at least one treatment with at least one antibiotic, said treatment being selected from the group consisting of:
oral administration of an efficient dose of at least one antibiotic at least after the application of the bone generating product to the patient;
oral administration of an efficient dose of at least one antibiotic at least prior the application of the bone generating product to the patient;
injection of an efficient dose of at least one antibiotic at least after the application of the bone generating product to the patient;
injection of an efficient dose of at least one antibiotic at least prior the application of the bone generating product to the patient;
administration of an efficient dose of at least one antibiotic at least for one day prior the application of the bone generating product and at least for one day after the application of the bone generating product to the patient.
